(19) United States
(12) Patent Application Publication
(10) Pub. No.: US 2002/0137105 A1
(43) Pub. Date: Sep. 26, 2002

(54) Method of evaluating aflatoxin exposure

(76) Inventors: Mei-Hui Wang, Hsin Chu (TW); Chang-Yi Wu, Taipei (TW); Te-Wei Lee, Taipei (TW); Henton Huang, Tau Yen (TW); Gann Ting, Taipei (TW)

Correspondence Address: Morton J. Rosenberg, Rosenberg, Klein & Lee, Suite 101, 3458 Ellicott Center Drive, Ellicott City, MD, 21043, US

(21) Appl. No.: 10/041,478

(22) Filed: Jan. 10, 2002

Related U.S. Application Data
(62) Division of application No. 09/718,341, filed on Nov. 24, 2000.

(51) Int. Cl. G01N 33/53; G01N 33/542; G01N 33/537; G01N 33/543

(52) U.S. Cl. 435/7.9; 435/7.92

(57) ABSTRACT

The invention discloses one radioimmunoassay testing kit and method for detecting aflatoxin-albumin adducts. The radioimmunoassay testing kit combining with competitive inhibition radioimmunometric assay, could be used to quantitate the aflatoxin-albumin adducts in serum. The invention also discloses their clinical uses in rapid mass detection of the doses of aflatoxin exposure, which is one of risk factors of liver cancer.

### FIELD OF THE INVENTION

The present invention relates to a radioimmunoassay testing kit and a method, which is used to quantitate the aflatoxin-albumin adduct. In particular the present invention relates to routine uses in serum. The present invention also relates to a method to determine if there is aflatoxin exposure or not. The normal cutoff value is also discloses.

### BACKGROUND OF THE INVENTION

Aflatoxins are toxic metabolites produced by the fungal species Aspergillus flavus and Aspergillus parasiticus. Aflatoxin B1 (AFB 1) is the most toxic group. Experimental normal evidence that aflatoxin is carcinogenic, especially in hepatotoxicity. Using the TD50 values for rats developed by Gold et al (Cancer Res. 1993, 53: 9-11). AFB1, which TD50=9.3×10⁻⁴ mg/kg per day, is 1000 times more potent a carcinogen than benzo(a)pyrene. Recently, the International Agency for Research on Cancer (IARC) reported that there is sufficient evidence to classify aflatoxin B1 and mixtures of aflatoxins as Group 1 carcinogens in humans.

AFB1 requires microsomal oxidation to the reactive AFB1-8,9-epoxide (AFBO) to exert its hepatocarcinogenic effects, and the extent of covalent binding of AFBO to cellular RNA, DNA, protein or other macromolecules (IARC 1993;56:303).

In 1987, Sabbioni et al reported excreted RNA and DNA adducts only reflect the previous 24-48 hours of exposure, a protein-based dosimeter which reflects weeks of exposure history could add greatly to the interpretation of epidemiological data.

Hemoglobin has the longest biological half-life among the proteins common proposed for dosimetry, but it binds to AFB1 is very low. Serum albumin, in contrast, binds a large fraction of ingested AFB1 as a stable covalent adduct. In humans, the half-life for turnover of serum albumin is about 20 days, which leads to chronic exposure adducts levels during the past 1-2 months (Carcinogenesis 8: 819-824, 1987)

Methods for testing of aflatoxin-albumin adduct in serum are the same as those for testing of aflatoxin before 1996. They are such as high performance liquid chromatography, radioimmunoassay, enzyme-linked immunosobent assay. But it is often the case that the serum needs to be digested with protease and extracted in organic solvent. The pretreatment of the process of extraction and purification is tedious. It seems not convenient for clinical routine usage.

This invention discloses one method and a radioimmunoassay testing kit which would be used to test aflatoxin-albumin adduct in serum without pretreatment, which improves the disadvantage of pretreatment. Besides, this invention also discloses a testing method to evaluate the extent of aflatoxin exposure by calculate the concentration of aflatoxin B1-albumin adducts per unit albumin in serum, which is very useful to predict the risk of liver cancer in the future.

### SUMMARY OF THE INVENTION

It is therefore an object of the present invention to provide a more convenient method for testing aflatoxin-albumin adducts. Serum needs not to be pretreated with extraction or enzyme digestion prior to test. There is no any enhancement or inhibition interferent in the serum matrix for this method.

It is therefore another object to the present invention to supply a new radioimmunoassay testing kit for accurately quantitating the aflatoxin-albumin adduct. The accuracy is 100.9±14%.

It is therefore another object of the present invention to provide a method for evaluating the extent of aflatoxin exposure, and its clinical uses to predict the risk of liver cancer. Besides, it discloses the relationship of aflatoxin exposure and hepatocarcinoma.

### BRIEF DESCRIPTION OF THE DRAWINGS

FIG. 1 is a graph showing the method of competitive inhibition radioimmunometric assay.

FIG. 2 is a graph showing the radioimmunoassay testing kit for detection of aflatoxin-albumin adducts.

FIG. 3 is a graph showing there is no matrix effect in the dose response curve for testing of aflatoxin-albumin adducts.

FIG. 4 is a graph showing the specificity of primary antibody against aflatoxin-albumun adduct. It exists no cross-reactivity to albumin.

FIG. 5 is a graph showing the detection range of the radioimmunoassay testing kit in this invention.

### DETAILED DESCRIPTION OF THE PREFERRED EMBODIMENTS

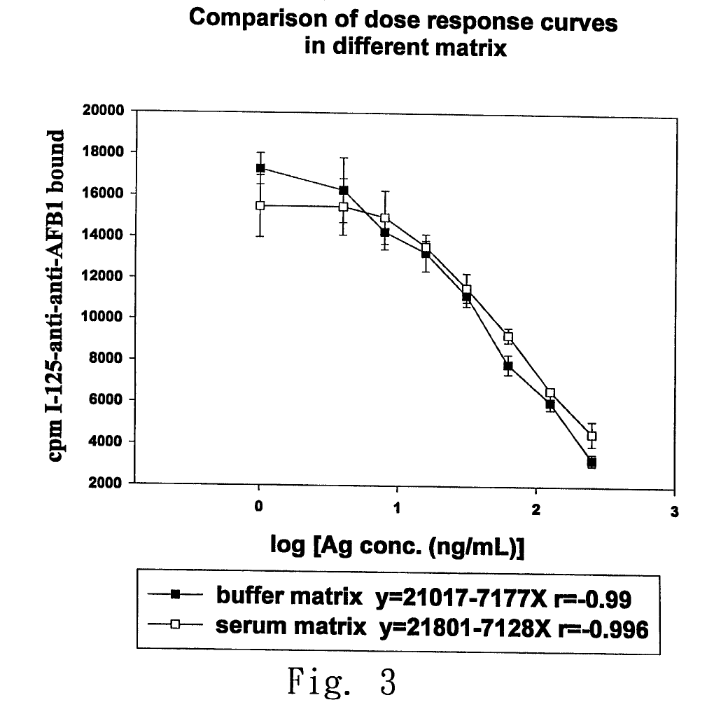

The present invention relates to a novel method for detecting aflatoxin-albumin adduct as an antigen using an competitive inhibition radioimmunometric assay, which comprises: providing primary antibody and testing serum into a microplate, then washing away material unbound onto microplate after enough incubation, then using I-125-labelled secondary antibody to detect the immune complex bound onto the microplate, wherein there are enough aflatoxin-albumin adducts bound on it, and both primary and secondary antibodies are polyclonal antibodies, which are derived by repeatly introducing aflatoxin-KLH into rabbits and by repeatly introducing rabbit immunoglobulin into animals except rabbit, respectively. The primary antibody has specificity against aflatoxin-albumin adducts, but has no cross reactivity to albumin. The secondary antibody has specificity to rabbit immunoglobulin but minimal cross-reaction with immunoglobulin from bovine and human. FIG. 3 shows there is no difference in dose responses curves for aflatoxin-albumin adducts in serum matrix and buffer matrix. It indicates there is no interferent in serum materials to enhance or inhibit the immune complex formation by primary antibody and aflatoxin-albumin adducts.

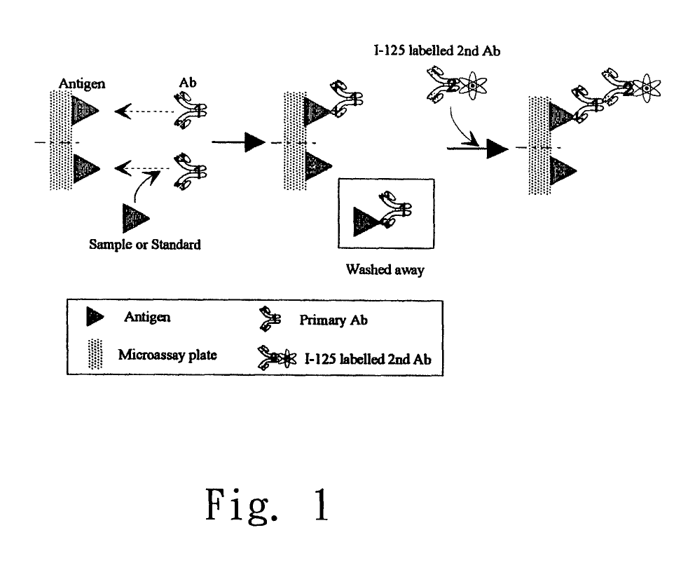

Futher the present invention relates to a kit for accurate testing an unknown amount of aflatoxin-albumin adducts in serum, which is unnecessary to be pretreated, wherein it contains a 6 mg/mL primary antibody with specificity to aflatoxin-albumin, and the working dilution is 1:2000, and wherein it contains a I-125-labelled secondary immunoglobulin with 5-20 uCi/ug, which is used to detect the immune complex formation by primary antibody and aflatoxin-albumin adducts, which working dilution is 1:5000, and wherein it contains standards of aflatoxin-albumin adducts, which is from 0 to 250 ng/mL and wherein it contains one solid phase, which is a microplate with enough aflatoxin-albumin adducts bound on it. Combing the method of FIG. 1, this kit could be used to test aflatoxin-albumin adducts in serum. The accuracy is 100±14% (Table 1).

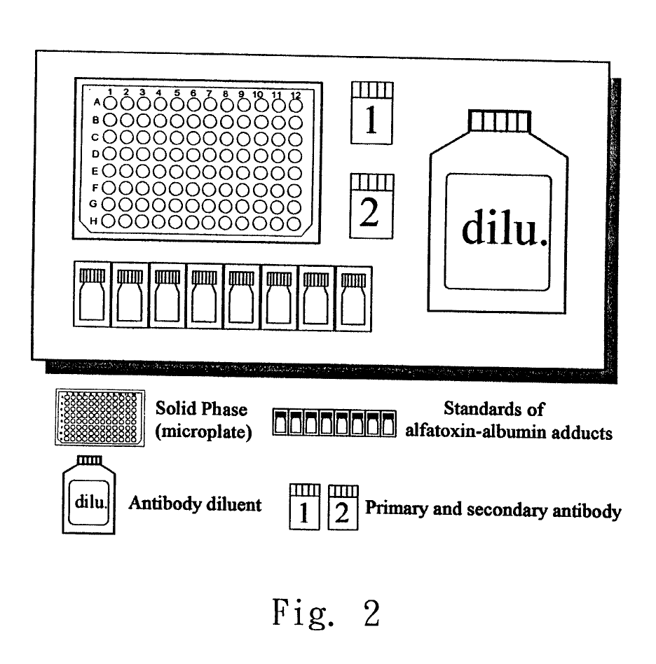

Finally the present invention relates to a method for evaluation of the dose of aflatoxin exposure, wherein aflatoxin-albumin testing only could not predict the risk of liver cancer, but testing for ng aflatoxin-albumin adducts per mg albumin is statistically clinically correlated with liver cancer, and wherein we set 5.32 ng aflatoxin-albumin adduct per mg albumin as normal cutoff value, and wherein there are 7.97 folds higher risk in people beyond this cutoff value than normal healthy persons, and wherein the aflatoxin-albumin adducts are testing by kit of FIG. 2 and method FIG. 1, and wherein albumin is testing by 628 nm absorption, which is the maximun absorption wavelength peaks of BCG (bromocresolgreen) and albumin complex.

### EXAMPLE 1

### Dose Response Curves of Aflatoxin-albumin Adducts in Different Matrix

Aflatoxin-albumin adduct standards in buffer matrix or serum matrix are adding to microplate respectively, wherein their standard range is from 0 to 250 ng/mL. Following the steps of FIG. 1, both of them are incubated with specific primary antibody, after enough time, washing away the unbound materials on the microplate. The immune complex bound to microplate was determined by I-125-labelled secondary antibody. FIG. 3 indicates, there seems no differences between standards in buffer matrix and those in serum matrix, because of their parallel dose response curves. The results indicate there is no interferent in serum matrix for aflatoxin-albumin testing. So the pretreatment steps of serum, such as extraction or enzyme digestion, is unnecessary.

### EXAMPLE 2

### Study on Specificity of Primary Antibody

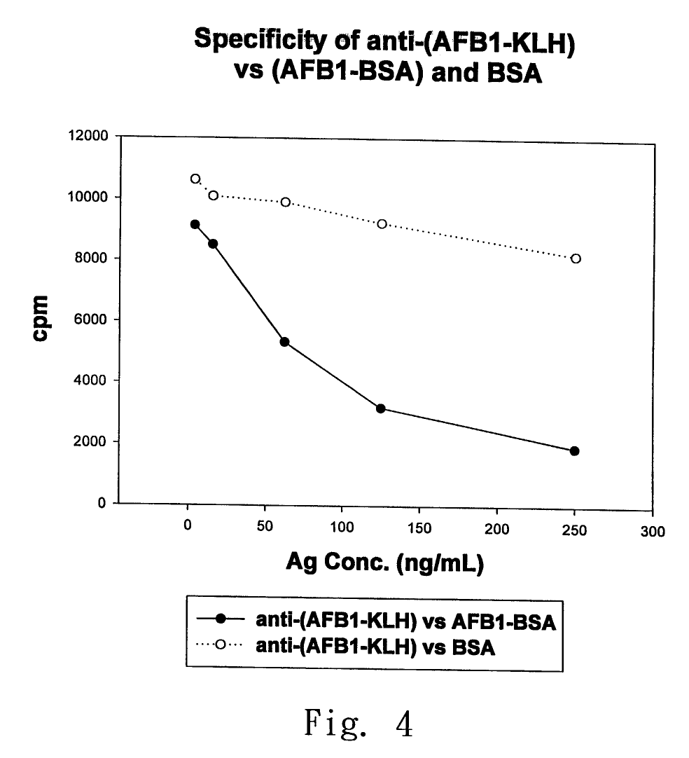

0-250 ng/mL standards of aflatoxin-albumin adducts or albumin is adding to microplate, respectively. Following the steps of FIG. 1, both of them are incubated with specific primary antibody, after enough time, washing away the unbound materials on the microplate. The immune complex bound to microplate was quantitated by I-125-labelled secondary antibody. FIG. 4 shows two dose response curves for aflatoxin-albumin adduct and albumin as antigens, respectively. There is no 50% inhibition of cross-reaction in the dose response curve of albumin as antigen, which indicates the primary antibody has specificity to aflatoxin-albumin adducts but has no cross-reaction with albumin.

### EXAMPLE 3

### Study on Detection Range of Aflatoxin-albumin Radioimmunoassay Testing Kit

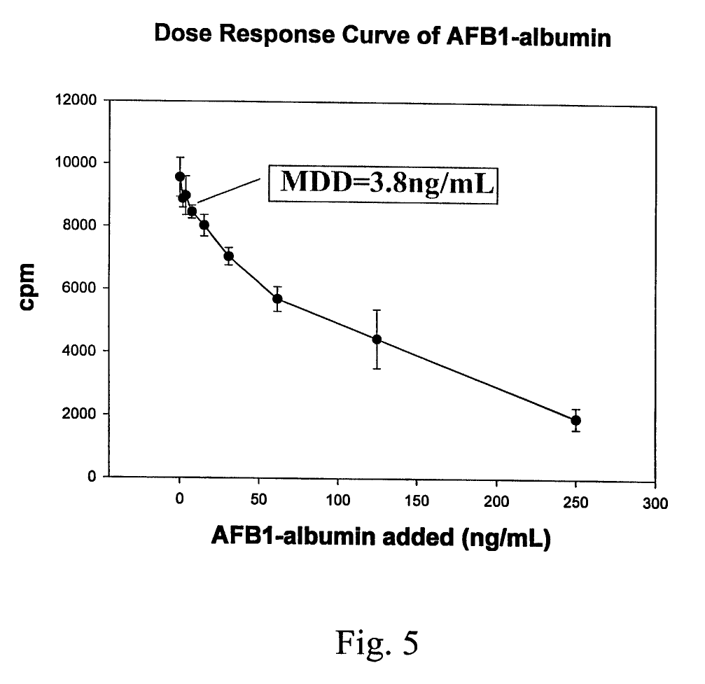

0-250 ng/mL standards of aflatoxin-albumin adducts, as standards in FIG. 2, are adding to microplate with triplicate. Following the steps of FIG. 1 by incubation enough time with specific primary antibody, washing away the unbound material. The bound immune complex were quantitated by I-125-labelled secondary antibody. FIG. 5 shows the performance of dose response curve by this kit. In this curve, the detection limit is 3.8 ng/mL, which is defined as the smallest concentration of an antigen that could be statistically distinguished from a zero level in an assay. The detection range is from 3.8 ng/mL to 250 ng/mL (FIG. 5) and there is linearity from 15.5 ng/mL to 125 ng/mL.

### EXAMPLE 4

### Study on Accuracy of Aflatoxin-albumin Radioimmunoassay Testing Kit

The accuracy is measured by the recovery of known concentration of aflatoxin-albumin adducts in serum matrix. Following the steps of FIG. 1, we got the detected concentration by comparing their cpms with the standard curve. The concentration added in the serum sample is 7.7 ng/mL and 31 ng/mL, respectively, and the concentration recovered are 8.3±1.4 ng/mL and 29.7±3.6 ng/mL, respectively. The recovery, which is called as accuracy and often calculated by concentration recovered divided by concentration added, is 100.9±14%.

TABLE 1
The accuracy of this aflatoxin-albumin
adducts radioimmunoassay testing kit

| | AFB1-albumin | recovery |
|---|---|---|
| AFB1-albumin added | measured | (%) |
| 31 ng/mL | 35 ng/mL | 113 |
| | 28 ng/mL | 90 |
| | 28.8 ng/mL | 93 |
| | 27 ng/mL | 87 |
| 7.7 ng/mL | 8.6 ng/mL | 112 |
| | 6.8 ng/mL | 88 |
| | 9.5 ng/mL | 123 |
| | | 100.9 ± 14.7% |

### EXAMPLE 5

### Case-control Study for Aflatoxin-albumin Testing

Choose two groups for case-control study. Both of their age mean were 53. Classified as “normals”, 16 people were identified as having normal albumin, GOT (glutamate oxaloacetic transaminase), GPT (glutamate pyruvate transaminase), abdominal sonagraphy, α-fetoprotein and no history of hepatoma. Classified as “hepatoma”, 16 patients were identified as having primary HCC (hepatocellular carcinoma) histologically confirmed by needle biopsy. All of the samples were testing for aflatoxin-albumin adducts by using kit of FIG. 2 and method of FIG. 1. The 95% confidence intervals of testing are 6.8-12.6 ng/mL for “normal” and 10.7-71.3 ng/mL for “hepatoma”, respectively. There is some overlap each other. See Table 2. But if testing for ng aflatoxin-albumin adduct per mg albumin, which albumin is tested by 628 nm absorption by complex with BCG, the 95% confidence intervals of testing are 1.4-2.8 ng/mg for “normal” and 2.9-19.7 ng/mg for “hepatoma”, respectively (Table 3). There is statistically significant difference each other. It indicates the highly correlation of aflatoxin exposure and liver cancer.

This invention indicates a method for quantitation of aflatoxin-albumin per mg albumin in sample could be used to evaluate the dose of aflatoxin exposure, which is highly correlated to liver cancer.

TABLE 2
Testing of aflatoxin-albumin adducts in 16 Normals and 16 Hepatomas

| | Normals | Hepatomas |
|---|---|---|
| | (ng/mL) | (ng/mL) |
| Mean | 9.7 | 41 |
| Mean of 95% confidence interval | 6.8˜12.6 | 10.7˜71.3 |

TABLE 3
Testing of ng aflatoxin-albumin adducts per
mg albumin in 16 Normals and 16 Hepatomas

| | Normals | Hepatomas |
|---|---|---|
| | (ng/mL) | (ng/mL) |
| Mean | 2.1 | 11.3 |
| Mean of 95% confidence interval | 1. 4˜2.8 | 2.9˜19.7 |

### EXAMPLE 6

### Normal Cutoff Value for Dose of Aflatoxin Exposure

To validate the normal cutoff value for dose evaluation of aflatoxin exposure, 195 patients were random chosen for testing of ng aflatoxin-albumin per mg albumin in serum. Classified as “normals”, 99 people were identified as having normal albumin, GOT, GPT, abdominal sonagraphy, α-fetoprotein and no history of hepatoma. Classified as “hepatoma”, 96 patients were identified as having primary HCC histologically confirmed by needle biopsy.

All of these samples were tested for ng aflatoxin-albumin per mg albumin in serum, which aflatoxin-albumin was tested by using kit of FIG. 2 and method of FIG. 1 , and albumin was tested by absorption at 628 nm by complex with BCG. The 95% confidence intervals of ng aflatoxin-albumin per mg albumin testing are 3.9-5.3 ng/mg and 11-17 ng/mg, respectively, which confirms again the highly correlation of aflatoxin exposure and liver cancer, in addition, the normal cutoff value is 5.32 ng/mg (Table 4).

TABLE 4
Testing of ng aflatoxin-albumin adducts per
mg albumin in 99 Normals and 96 Hepatomas

| | Normals | Hepatomas |
|---|---|---|
| | (ng/mL) | (ng/mL) |
| Mean | 4.6 | 14.0 |
| Mean of 95% confidence interval | 3.9˜5.3 | 11˜17 |

### EXAMPLE 7

### Risk of Liver Cancer if Dose of Aflatoxin Exposure is More Than 5.32 ng/mg

The same 195 study identified a group of 96 liver cancer cases with ages about 55 and a group of 99 age, sex, resident-matched healthy controls by random chosen. The main purpose of the study was to look at the effect of aflatoxin exposure on liver cancer risk. In this study, aflatoxin exposure “yes” was defined as exposure dose more than normal cutoff value. To display the data, a 2×2 table relating case-control status to aflatoxin exposure can be constructed for “hepatomas” and “controls”. The data are given in Table 5.

TABLE 5
Risk of liver cancer on aflatoxin exposure

disease

| exposure | Hepatomas | Normals | |
|---|---|---|---|
| yes | 70 | 25 | 95 |
| no | 26 | 74 | 100 |
| | 96 | 99 | 195 |

The odds ratio is 7.97 (4.2˜15.1).

In this case-control study, there are 70 people among 96 “hepatomas” aflatoxin exposure “yes”, but there are 25 people among 99 healthy “controls” aflatoxin exposure “yes”. The estimation of odds ratio was according to the Mantel-Haenzel's method. Based on Table 5, the odds ratio

OR=(70×74)/(25×26)=7.97, and its 95% confidence interval is

7.97×exp[±1.96√(1/70+1/74+1/26+1/25)]=4.2˜15.1

The risk to have the liver cancer is 7.97 measured by odds ratio for people with aflatoxin exposure, which is 7.97-fold higher than people with no aflatoxin exposure.

Although the present invention has been described with reference to the preferred embodiments, it will be understood that the invention is not limited to the details described thereof. Various substitutions and modifications have been suggested in the foregoing description, and others will occur to those of ordinary skill in the art. Therefore, all such substitutions and modifications are intended to be embraced within the scope of the invention as defined in the appended claims.

What is claimed is:

1. A method for testing aflatoxin-albumin adducts, which could be applied to detect serum without pretreatment, there is no interferent existing in serum to enhance or inhibit the conformation of the complex by the antibody and aflatoxin-albumin adducts, comprising the following steps:
(a) providing specific primary antibody and serum sample into a solid phase, and incubating enough time, then washing away the unbound immune complex onto the solid phase; and
(b) using I-125-secondary antibody to detect the immune complex bound left onto the solid phase.

2. The method of claim 1, wherein the solid phase is a microplate, which binds a known concentration of aflatoxin-albumin adducts as antigen onto it.

3. The method of claim 1, wherein the primary antibody is one polyclonal antibody specific to aflatoxin-albumin but no cross-reaction with albumin, wherein the antibody is derived by repeatedly introducing aflatoxin-KLH intro a rabbit so that the polyclonal antibody are provided in blood serum of the rabbit.

4. The method of claim 1, wherein the secondary antibody is another polyclonal antibody specific to primary antibody but no cross-reaction with immunoglobulins of bovine and human, wherein the antibody is derived by repeatly introducting rabbit immunoglobulin into animals except rabbits, so that the polyclonal antibody are provided in the blood serum of that animal.

5. A radioimmunoassay testing kit for accurate test of aflatoxin-albumin adducts by using the method of claim 1, which comprises:
(a) a primary antibody specific to aflatoxin-albumin, herein there is no interferent in serum matrix to enhance or inhibit the formation of immune complex;
(b) a I-125-labelled secondary antibody, which is specific to immune complex conformed by the primary antibody and aflatoxin-albumin adducts;
(c) standards of aflatoxin albumin adducts which could be used to setup the standard dose response curve for aflatoxin-albumin testing; and
(d) a solid phase which binds the known concentration of aflatoxin-albumin adducts as the antigen.

6. A method for evaluation of the dose of aflatoxin exposure, herein the dose of aflatoxin exposure is measuring by testing the quantity of aflatoxin-albumin adduct per mg albumin in serum, people with aflatoxin exposure have 7.97-fold higher risk to become liver cancer than people with no aflatoxin exposure, which comprises:
(1) the aflatoxin-albumin is testing with the kit of claim 5, by the following manner,
(a) providing specific primary antibody and serum sample into a solid phase, and incubating enough time, then washing away the unbound immune complex onto the solid phase;
(b) using I-125-secondary antibody to detect the immune complex bound left onto the solid phase; and
(2) the albumin is testing with 628 nm absorption by formation of albumin-BCG complex.
